(19) United States
(12) Patent Application Publication
(10) Pub. No.: US 2009/0298115 A1
(43) Pub. Date: Dec. 3, 2009

(54) Fluorescent Gold Nanocluster and Method for Forming the Same

(75) Inventors: Walter Hong-Shong Chang, Tao-Yuan (TW); Cheng-An Lin, Tao-Yuan (TW); Ting-Ya Yang, Tao-Yuan (TW); Chih-Hsien Lee, Tao-Yuan (TW); Ralph A. Sperling, Eltville am Rhein (DE); Wolfgang J. Parak, Marburg (DE)

Correspondence Address: WPAT, PC;INTELLECTUAL PROPERTY ATTORNEYS, 7225 BEVERLY ST., ANNANDALE, VA, 22003, US

(73) Assignee: CHUNG YUAN CHRISTIAN UNIVERSITY, Tao-Yuan (TW)

(21) Appl. No.: 12/425,751

(22) Filed: Apr. 17, 2009

Related U.S. Application Data
(60) Provisional application No. 61/057,001, filed on May 29, 2008.

(30) Foreign Application Priority Data
Aug. 29, 2008 (TW) 097133042

(51) Int. Cl. C12Q 1/02

(52) U.S. Cl. 435/29; 977/774

(57) ABSTRACT

The present invention discloses a fluorescent gold nanocluster, comprising: a dihydrolipoic acid ligand (DHLA) on the surface thereof, wherein the fluorescent gold nanocluster generates fluorescence by the interaction between the dihydrolipoic acid ligand and the nanocluster and the particle diameter of the fluorescent gold nanocluster is between 0.5 nm and 3 nm, wherein the wavelength of the emission fluorescence of the fluorescent gold nanocluster is between 400 nm and 1000 nm. In addition, the fluorescent gold nanocluster is used as bioprobes and/or applied in fluorescent biological label, clinical image as contrast medium, clinical detection, clinical trace, and clinical treatment etc.

### BACKGROUND OF THE INVENTION

1. Field of the Invention

The present invention generally relates to a gold nanocluster, and more particularly to a water-soluble fluorescent gold nanocluster and preparation method thereof.

2. Description of the Prior Art

In modern biological analysis, various kinds of organic dyes are used. However, with each passing year, more flexibility is being required of these dyes, and the traditional dyes are often unable to meet the expectations. To this end, semiconductor quantum dots have quickly filled in the role, being found to be superior to traditional organic dyes on several counts, one of the most immediately obvious being brightness (owing to the high quantum yield) as well as their stability (much less photodestruction).

The use of semiconductor quantum dots for highly sensitive cellular imaging has seen major advances over the past decade. The improved photostability of semiconductor quantum dots for example, allows the acquisition of many consecutive focal-plane images that can be reconstructed into a high-resolution three-dimensional image. Another application that takes advantage of the extraordinary photostability of quantum dot probes is the real-time tracking of molecules and cells over extended periods of time.

Semiconductor quantum dots have also been employed for in vitro imaging of pre-labeled cells. The ability to image single-cell migration in real time is expected to be important to several research areas such as embryogenesis, cancer metastasis, stem-cell therapeutics, and lymphocyte immunology.

But there is a remaining issue with semiconductor quantum dot probes containing toxic ions, such as Cadmium and Lead. For this reason, we have been used fluorescent gold nanoclusters, so-called gold-quantum dots, instead of semiconductor quantum dots, wherein gold-quantum dots is nontoxic, having biocompatibility and high fluorescence quantum yield. Moreover, it is confirmed that gold-quantum dots is able to process different fluorescence colors by changing size thereof.

However, it is really difficult to synthesize gold-quantum dots. Gold-quantum dots are from PAMAM-encapsulated Au generally, wherein the PAMAM dendrimer is costly and gold-quantum dots are unable to be mass production at once.

Therefore, in view of the above mentioned problems, a novel method for synthesize gold-quantum dots is an important research topic in industry.

### SUMMARY OF THE INVENTION

According to the above, the present invention provides a fluorescent gold nanocluster to fulfill the requirements of this industry.

One object of the present invention is to discloses a fluorescent gold nanocluster, comprising: a dihydrolipoic acid ligand (DHLA) on the surface thereof, wherein the fluorescent gold nanocluster generates fluorescence by the interaction between the dihydrolipoic acid ligand and the nanocluster and the particle diameter of the fluorescent gold nanocluster is between 0.5 nm and 3 nm.

Another object of the present invention is to discloses a fluorescent gold nanocluster matrix, comprising: a plurality of gold nanoclusters piled up regularly wherein the particle diameter of the gold nanocluster is between 0.5 nm and 3 nm; the surface of the gold nanocluster comprises alkanethiol ligand(s); the gold nanoclusters are piled up due to the interaction between the alkanethiol ligands on the surface thereof to form the fluorescent gold nanocluster matrix; and the fluorescent gold nanocluster matrix has the fluorescence property by aggregating the gold nanoclusters.

The other object of the present invention is to disclose a method for forming a metal nanocluster. At first, providing a mixture solution that comprises a first metal precursor, a surfactant, a reductant, and a solvent wherein a reduction reaction is performed in the mixture solution to form a metal nanoparticle. Following that, adding a second metal precursor after the metal nanoparticle is formed, to have the number of particles of the second metal precursor be more than the total number of the metal nanoparticles, wherein the concentration difference between the metal nanoparticle and the second metal precursor results in a non-equilibrium coexistence system and then the metal nanoparticle breaks down to metal nanoclusters with a smaller particle diameter so as to form an equilibrium system.

### BRIEF DESCRIPTION OF THE DRAWINGS

FIG. 1 is the absorption spectrum of the gold nanoparticles and the gold nanoclusters of the first example of the present invention;

FIG. 2 is the absorption, the photoluminescence (PL), and the photoluminescence excitation (PLE) spectrums of the AuNC-DHLA nanoclusters of the first example of the present invention;

FIG. 3 is the schematic diagram of the conjugation of the fluorescent gold nanoclusters with biomolecules of the first example of the present invention;

FIG. 4 is the PLE and the PL spectrums of the AuNC-DDT nanoclusters of the second example of the present invention; and

FIG. 5 is the absorption spectrums of the gold nanoparticles and the AuNC-DDT nanoclusters of the second example of the present invention.

### DESCRIPTION OF THE PREFERRED EMBODIMENTS

What is probed into the invention is a fluorescent gold nanocluster and method for forming the same. Detail descriptions of the structure and elements will be provided in the following in order to make the invention thoroughly understood. Obviously, the application of the invention is not confined to specific details familiar to those who are skilled in the art. On the other hand, the common structures and elements that are known to everyone are not described in details to avoid unnecessary limits of the invention. Some preferred embodiments of the present invention will now be described in greater detail in the following specification. However, it should be recognized that the present invention can be practiced in a wide range of other embodiments besides those explicitly described, that is, this invention can also be applied extensively to other embodiments, and the scope of the present invention is expressly not limited except as specified in the accompanying claims.

Having summarized various aspects of the present invention, reference will now be made in detail to the description of the invention as illustrated in the drawings. While the invention will be described in connection with these drawings, there is no intent to limit it to the embodiment or embodiments disclosed therein. On the contrary the intent is to cover all alternatives, modifications and equivalents included within the spirit and scope of the invention as defined by the appended claims.

It is noted that the drawings presents herein have been provided to illustrate certain features and aspects of embodiments of the invention. It will be appreciated from the description provided herein that a variety of alternative embodiments and implementations may be realized, consistent with the scope and spirit of the present invention. It is also noted that the drawings presents herein are not consistent with the same scale. Some scales of some components are not proportional to the scales of other components in order to provide comprehensive descriptions and emphasizes to this present invention.

The first embodiment of the present invention discloses a fluorescent gold nanocluster, comprising: a dihydrolipoic acid ligand (DHLA) on the surface thereof, wherein the fluorescent gold nanocluster generates fluorescence by the interaction between the dihydrolipoic acid ligand and the nanocluster and the particle diameter of the fluorescent gold nanocluster is between 0.5 nm and 3 nm. In addition, the wavelength of the excited fluorescence of the fluorescent gold nanocluster is between 400 and 1000 nm.

In one preferred example of the first embodiment, the fluorescent gold nanocluster further comprising: a spacer, one end of which is bonded to the dihydrolipoic acid ligand (DHLA) and the other end of which has a specific moiety. The spacer comprises an oligomer or polymer. In addition, the specific moiety comprises one substance selected from the group consisting of the following or combination thereof: chemical functional group, crosslinking molecule, saccharide, fluorescent molecule, paramagnetic molecule, bio-molecule, and drugs.

The oligomer or polymer comprises one substance selected from the group consisting of the following or combination thereof: polyols, polyether polyols, polyester polyols, polycarbonate polyols, polycaprolactone polyols, polyacrylate polyols, polyethylene glycol (PEG), dextran, and copolymers thereof.

In another preferred example of the first embodiment, the fluorescent gold nanocluster further comprising: a spacer bonded to the dihydrolipoic acid ligand (DHLA) wherein the spacer has a specific moiety inherently, wherein the spacer comprises one substance selected from the group consisting of the following or combination thereof: chemical functional group, crosslinking molecule, saccharide, fluorescent molecule, paramagnetic molecule, bio-molecule, and drugs.

As described in the above, the fluorescent gold nanoclusters are used as bioprobes and/or applied in fluorescent biological label, clinical image as contrast medium, clinical detection, clinical trace, and clinical treatment.

The second embodiment of the present invention discloses a fluorescent gold nanocluster matrix, comprising: a plurality of gold nanoclusters piled up regularly wherein the particle diameter of the gold nanocluster is between 0.5 nm and 3 nm; the surface of the gold nanocluster comprises alkanethiol ligand(s); the gold nanoclusters are piled up due to the interaction between the alkanethiol ligands on the surface thereof to form the fluorescent gold nanocluster matrix; and the fluorescent gold nanocluster matrix has the fluorescence property by aggregating the gold nanoclusters. In addition, the wavelength of the excited fluorescence of the fluorescent gold nanocluster matrix is between 400 nm and 1000 nm.

In one preferred example of the second embodiment, the fluorescent gold nanocluster matrix further coating a spacer on the surface of nanoclusters matrix wherein one end of the spacer is bonded to the alkanethiol and the other end of the spacer has a specific moiety. The spacer comprises an amphiphilic polymer or oligomer. In addition, the specific moiety comprises one substance selected from the group consisting of the following or combination thereof: chemical functional group, cross-linking molecule, saccharide, fluorescent molecule, paramagnetic molecule, bio-molecule, and drugs.

Moreover, the amphiphilic polymer or oligomer comprises one substance selected from the group consisting of the following or combination thereof: poly(maleic anhydride) (PMA), Poly(maleic anhydride-alt-1-octadecene) (PMAO), polyacrylic acid (PAA), and derivatives thereof.

In another preferred example of the second embodiment, the fluorescent gold nanocluster matrix further coated a spacer on the surface of nanoclusters matrix, wherein one end of the spacer bonded to the alkanethiol, wherein the spacer has a specific moiety inherently. In addition, the spacer comprises one substance selected from the group consisting of the following or combination thereof: chemical functional group, crosslinking molecule, saccharide, fluorescent molecule, paramagnetic molecule, bio-molecule, and drugs.

As described in the above, the fluorescent gold nanocluster matrix is used as bioprobes and/or applied in fluorescent biological label, clinical image as contrast medium, clinical detection, clinical trace, and clinical treatment.

The third embodiment of the present invention discloses a method for forming a metal nanocluster. At first, providing a mixture solution that comprises a first metal precursor, a surfactant, a reductant, and a solvent wherein a reduction reaction is performed in the mixture solution to form a metal nanoparticle wherein the metal nanoparticle comprises a property of surface plasmon absorption.

Following that, adding a second metal precursor after the metal nanoparticle is formed, to have the number of particles of the second metal precursor be more than the total number of the metal nanoparticles, wherein the concentration difference between the metal nanoparticle and the second metal precursor results in a non-equilibrium coexistence system and then the metal nanoparticle breaks down to metal nanoclusters with a smaller particle diameter so as to form an equilibrium system. In addition, the particle diameter of the metal nanocluster is between 1 nm and 4 nm.

Besides, the first metal precursor and the second metal precursor are the same or different wherein the above mentioned precursors are selected from the group consisting of the following: AuCl3, HAuCl4, AuBr3, and HAuBr4.

In addition, the surfactant is selected from the group consisting of the following or combination thereof: didodecyldimethylammonium bromide (DDAB), tetraoctylammonium bromide (TOAB), cetyltrimethyl ammonium bromide (CTAB), tetrabutylammonium bromide (TBAB) and etc. The reductant is selected from the group consisting of the following or combination thereof: tetrabutylammonium borohydride (TBAB), NaBH4, and ascorbic acid. Moreover, the solvent is selected from the group consisting of the following or combination thereof: toluene and chloroform.

On the other hand, a ligand-binding reaction is performed, after the metal nanocluster is formed, to bind a ligand to the surface of the metal nanocluster so as to form a ligand-capped metal nanocluster, wherein the ligand is selected from the group consisting of the following: dihydrolipoic acid (DHLA), dodecanethiol (DDT), Bis(p-sulfonatophenyl)phenylphosphine (BSPP), and triphenylphosphine etc.

Furthermore, the ligand-binding reaction is a thiol-related ligand binding reaction to bind a thiol-related ligand to the surface of the metal nanocluster so as to form a thiol-capped metal nanocluster, wherein the thiol-related ligand is selected from the group consisting of the following: dihydrolipoic acid (DHLA), dodecanethiol (DDT), meso-2,3-dimercaptosuccinic acid (DMSA), glutathione (GSH), and 1,6-hexanedithiol. Forthermore, the thiol-capped metal nanocluster is a fluorescent metal nanocluster wherein the particle diameter of the fluorescent metal nanocluster is between 0.5 nm and 3 nm.

Subsequently, performing a functional coating reaction after the fluorescent metal nanocluster is formed, to have the fluorescent metal nanocluster comprise a functional group, wherein the functional group of the functional coating reaction is selected from the group consisting of the following: chemical functional group, crosslinking molecule, saccharide, fluorescent molecule, paramagnetic molecule, bio-molecule, and drugs, wherein the functional coating reaction is a bioconjugation reaction.

As described in the above, the fluorescent metal nanocluster is used as bioprobes and/or applied in fluorescent biological label, clinical image as contrast medium, clinical detection, clinical trace, and clinical treatment.

### Example 1

### Fluorescent Gold Nanoclusters

### (A) Synthesis of the Fluorescentgold Nanoclusters (AuNC@DHLA)

### (I) Synthesis of Gold Nanocluster

Appropriate amounts of DDAB are dissolved in toluene to produce a stock solution with a concentration of 100 mM concentration. Then gold precursor solution (25 mM) is prepared by dissolving appropriate amounts of gold (III) chloride (AuCl₃ or HAuBr₄) in the DDAB solution. Then, 1 mL of fresh-prepared TBAB solution (100 mM in the DDAB stock solution) is mixed with 0.625 mL decanoic acid solution under vigorous stirring and 0.8 mL precursor solution such as gold (III) chloride solution is injected so as to form a dark-red solution of gold nanoparticles. The gold nanoparticles are collected by methanol-induced agglomeration, i.e. by adding excess methanol until obtaining a blue-purple cloudy solution. Free surfactants, reduction agents and other smaller nanoparticles are removed by discarding the supernatants of solution upon centrifugation (2500 rpm, 30 min).

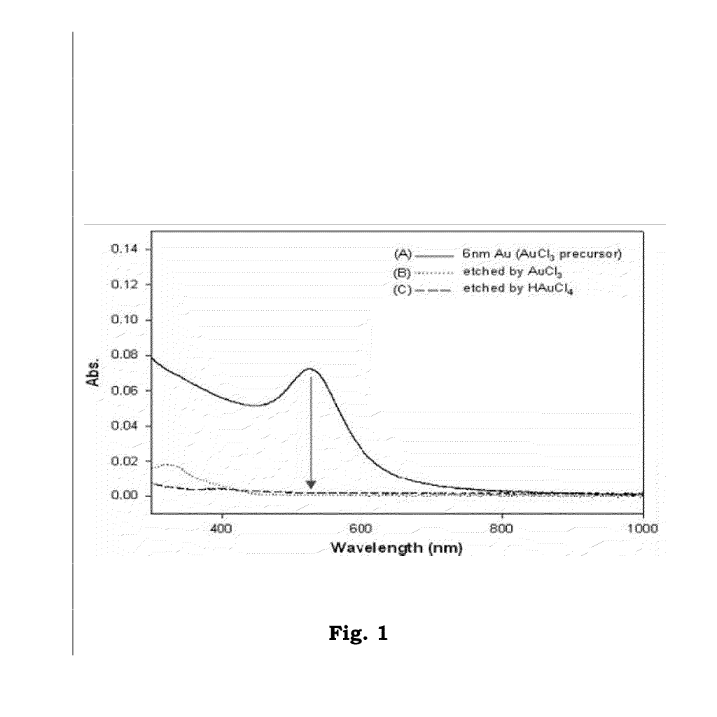

The wet precipitants of nanoparticles are then re-dissolved in DDAB solution, giving a dark blood-red color. Upon adding several drops of gold precursor solution while stirring them, the solution color turns from dark to yellowish transparency. All the gold nanoparticles are etched into smaller clusters due to the vanishing of surface plasmon absorption around 520 to 530 nm, but the gold nanoclusters do not have this property. Referring to FIG. 1, (A) gold nanoparticles using AuCl₃ as the first gold precursor; (B) gold nanoclusters using AuCl₃ as the second gold precursor; (C) gold nanoclusters using HAuBr₄ as the second gold precursor.

### (II) Ligand Exchange to the Gold Nanocluster

0.0322 g of TBAB powder and 2.5 mL of DDAB solution are mixed until no solid powders. 0.052 g of lipoic acid powders is added into the solution until no bubbling in order to reduce the lipoic acid into dihydrogenlipoic acid (DHLA). In order to avoid the residue activity of lipoic acid, another TBAB powders is loaded with excess to the reduced lipoic a c id mixture and stirring until no bubbling. Then, 2.5 mL of gold nanoclusters solution and dihydrogenlipoic acid (DHLA) are mixed by stirring them. When the fluorescent gold nanoclusters are formed, the color of solution turns to a yellow-brown cloudy solution.

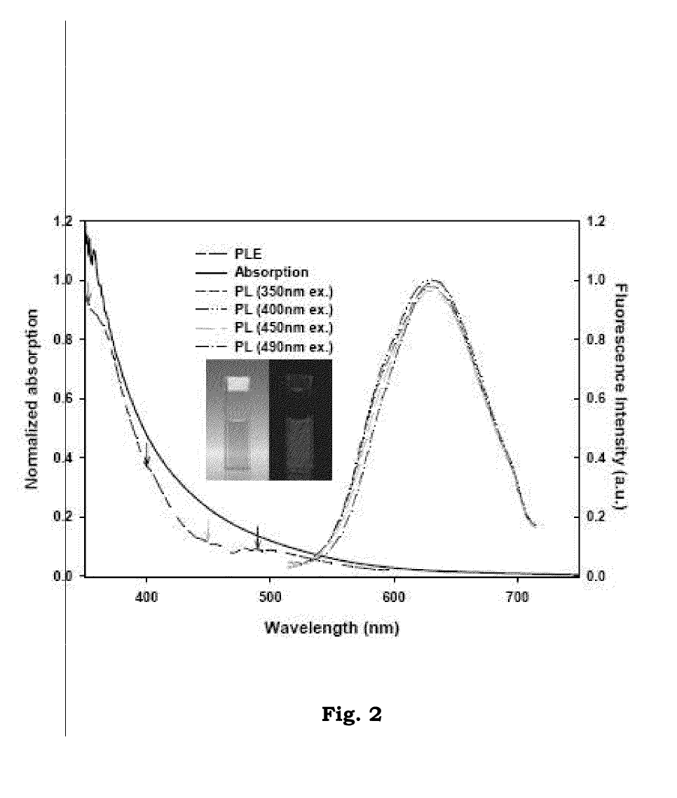

FIG. 2 shows the absorption, the photoluminescence (PL), and the photoluminescence excitation (PLE) spectrum.

### (B) Conjugating Biomolecular to Fluorescent Gold Nanoclusters

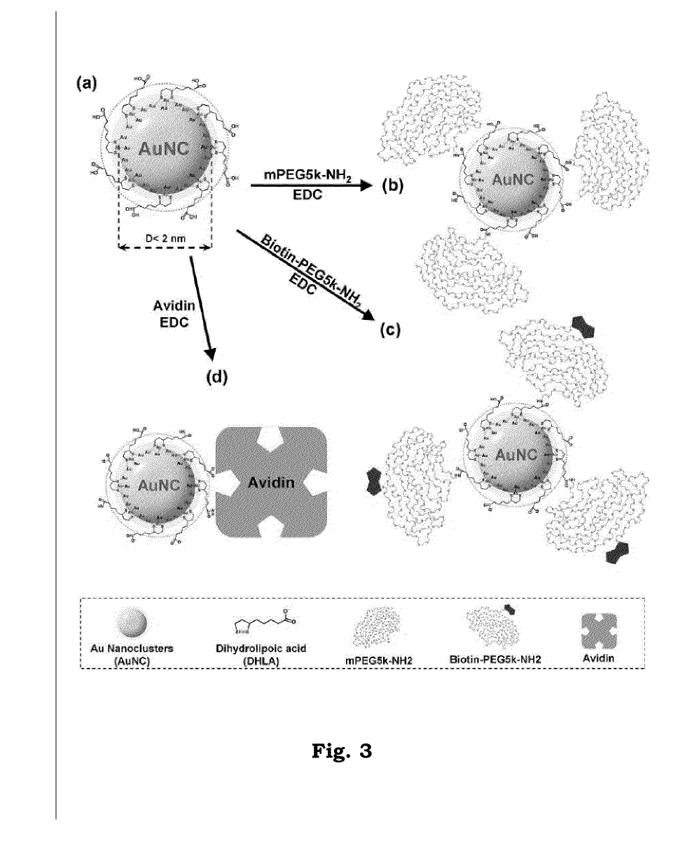

10 micro-liter of gold nanocluster solution and 10 micro-liter X-PEG-amine (3 mM in ddH₂O) are mixed, giving a mixing solution. Then, added 1-ethyl-3-(3-dimethylaminopropyl)carbodiimide (EDC, 8 mM in ddH₂O) into the mixing solution and shake it for two hours. FIG. 3 shows the schematic diagram of grating bio-molecular on fluorescent gold nanoclusters. Furthermore, the modification of fluorescent gold nanoclusters are selected by gel electrophoresis (2% agarose, 75 V), and are put into the SBB (sodium borate buffer, pH=9) via centrifugation of 100 KDa molecular sieve.

### Example 2

### fluorescent Au Nanocluster Matrix

### (A) Synthesis of the Fluorescent Gold Nanocluster Matrix

### (I) Ligand Exchange to the Fluorescent Gold Nanocluster Matrix

Provide a gold nanoclusters solution formed by the method demonstrated in example 1 (I). The above-mentioned gold nanoclusters solution are dropped slowly into dodecanethiol (DDT) solution while stirring them for ligand exchange on the surface of the nanocluster. After the solution is stirred for an hour, the solution becomes a cloudy solution and the aggregation of fluorescent gold nanoclusters are formed.

### (B) Bioconjugation for Fluorescent Gold Nanocluster Matrix

200 micro-liter of fluorescent gold nanoclusters solution and 200 micro-liter of galactose solution (80 mM in ddH₂O) are mixed. Then, add 1-ethyl-3-(3-dimethylaminopropyl)carbodiimide (EDC, 30 mM in ddH₂O) as a cross-linking agent, and provide a shacking process for two hours. Amide linkage is formed by EDC bounding at the amine of galactose so that galactose is grated on the surface of the fluorescent gold nanoclusters. The residued of galactose is removed via centrifugation of 100 KDa molecular sieve.

20 micro-liter of the above-mentioned fluorescent gold nanoclusters and 20 micro-liter of RCA120 (1 mg/mL) are mixed for twenty minutes to test the galactose grated on the surface of the fluorescent gold nanoclusters.

Furthermore, the modification of fluorescent gold nanoclusters are selected by gel electrophoresis (2% agarose, 75 V), and are put into the SBB (sodium borate buffer, pH=9) via centrifugation of 100 KDa molecular sieve.

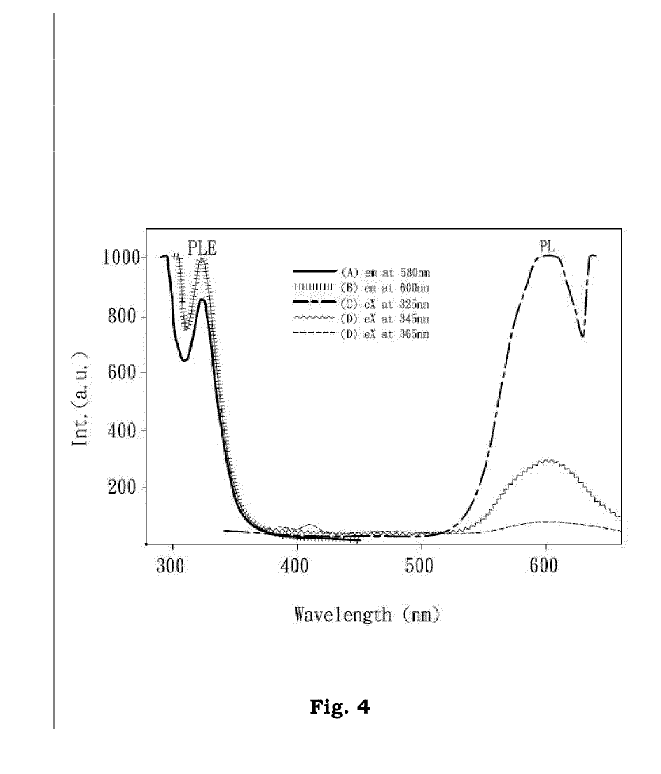

The more Au—S bonds are produced, the stronger intensity of fluorescence of gold nanoclusters becomes. The Au-DDT fluorescent gold nanoclusters are treated with 325 nm, 345 nm, and 365 nm light and produce red fluorescence at 600 nm. The position of the pick retains at the same place, so it is not the ordinary scattering. As shown in FIG. 4, the Au-DDT is treated with 325 nm light, and the largest intensity of red fluorescence at 600 nm appeares.

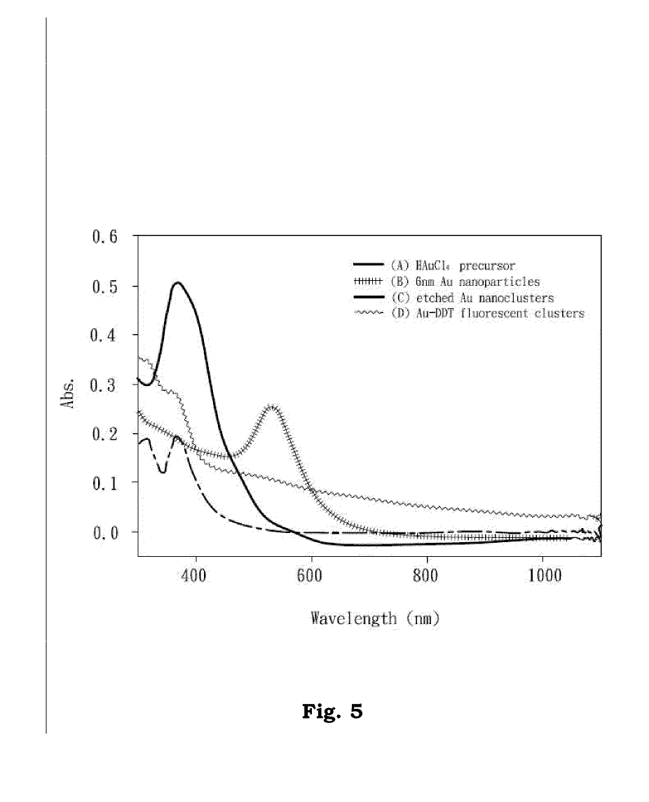

As shown in FIG. 5, HAuCl4 precursor has a specific absorption peak at 370 nm, and it is treated with TBAB to produce 6 nm gold nanoparticles which have surface plasmon resonance absorption peak at 520 nm. The gold nanoparticles are broken into gold nanoclusters via adding HAuCl₄ precursor. The 520 nm absorption peak disappears. It means that the diameter of gold nanoclusters is less than 5 nm. In addition to the 370 nm peak, a 310 nm peak appears. The hydrophobic chain makes the gold nanoclusters self-assemble and the area of absorption peak increases. The absorption spectrum of (A) HAuCl₄ precursor (0.625 mM), (B) gold nanoparticles, (C) gold nanoclusters, and (D) AuNC-DDT fluorescent gold nanoclusters are shown in FIG. 5.

Obviously many modifications and variations are possible in light of the above teachings. It is therefore to be understood that within the scope of the appended claims the present invention can be practiced otherwise than as specifically described herein. Although specific embodiments have been illustrated and described herein, it is obvious to those skilled in the art that many modifications of the present invention may be made without departing from what is intended to be limited solely by the appended claims.

1. A fluorescent gold nanocluster, comprising: a dihydrolipoic acid ligand (DHLA) on the surface thereof, wherein said fluorescent gold nanocluster generates fluorescence by the interaction between said dihydrolipoic acid ligand and said nanocluster and the particle diameter of said fluorescent gold nanocluster is between 0.5 nm and 3 nm.

2. The nanocluster according to claim 1, further comprising: a spacer, one end of which is bonded to said dihydrolipoic acid ligand (DHLA) and the other end of which has a specific moiety.

3. The nanocluster according to claim 2, wherein said spacer comprises an oligomer or polymer.

4. The nanocluster according to claim 2, wherein said oligomer or polymer comprises one substance selected from the group consisting of the following or combination thereof: polyols, polyether polyols, polyester polyols, polycarbonate polyols, polycaprolactone polyols, polyacrylate polyols, polyethylene glycol (PEG), dextran, and copolymers thereof.

5. The nanocluster according to claim 2, wherein said specific moiety comprises one substance selected from the group consisting of the following or combination thereof: chemical functional group, crosslinking molecule, saccharide, fluorescent molecule, paramagnetic molecule, bio-molecule, and drugs.

6. The nanocluster according to claim 1, further comprising: a spacer bonded to said dihydrolipoic acid ligand (DHLA) wherein said spacer has a specific moiety inherently.

7. The nanocluster according to claim 6, wherein said spacer comprises one substance selected from the group consisting of the following or combination thereof: chemical functional group, crosslinking molecule, saccharide, fluorescent molecule, paramagnetic molecule, bio-molecule, and drugs.

8. The nanocluster according to claim 1, wherein the wavelength of the excited fluorescence of said fluorescent nanocluster is between 400 and 1000 nm.

9. The nanocluster according to claim 1, wherein said nanocluster is used as bioprobes and/or applied in fluorescent biological label, clinical image as contrast medium, clinical detection, clinical trace, and clinical treatment.

10. A fluorescent gold nanocluster matrix, comprising: a plurality of gold nanoclusters piled up regularly wherein the particle diameter of said gold nanocluster is between 0.5 nm and 3 nm; the surface of said gold nanocluster comprises alkanethiol ligand(s); said gold nanoclusters are piled up due to the interaction between said alkanethiol ligands on the surface thereof to form said fluorescent gold nanocluster matrix; and said fluorescent gold nanocluster matrix has the fluorescence property by aggregating said gold nanoclusters.

11. The matrix according to claim 10, further coating a spacer on the surface thereof wherein one end of said spacer is bonded to said alkanethiol and the other end of said spacer has a specific moiety inherently.

12. The matrix according to claim 11, wherein said spacer comprises an amphiphilic polymer or oligomer.

13. The matrix according to claim 12, wherein said amphiphilic polymer or oligomer comprises one substance selected from the group consisting of the following or combination thereof: poly(maleic anhydride) (PMA), Poly(maleic anhydride-alt-1-octadecene) (PMAO), polyacrylic acid (PAA), and derivatives thereof.

14. The matrix according to claim 12, wherein said specific moiety comprises one substance selected from the group consisting of the following or combination thereof: chemical functional group, crosslinking molecule, saccharide, fluorescent molecule, paramagnetic molecule, bio-molecule, and drugs.

15. The matrix according to claim 10, further coating a spacer on the surface thereof wherein one end of said spacer bonded to said alkanethiol wherein said spacer has a specific moiety.

16. The matrix according to claim 15, wherein said spacer comprises one substance selected from the group consisting of the following or combination thereof: chemical functional group, crosslinking molecule, saccharide, fluorescent molecule, paramagnetic molecule, bio-molecule, and drugs.

17. The matrix according to claim 10, wherein the wavelength of the excited fluorescence of said fluorescent gold nanocluster matrix is between 400 and 1000 nm.

18. The matrix according to claim 10, wherein said fluorescent gold nanocluster matrix is used as bioprobes and/or applied in fluorescent biological label, clinical image contrast medium, clinical detection, clinical trace, and clinical treatment.

19. A method for forming a metal nanocluster, the method comprising:
providing a mixture solution that comprises a first metal precursor, a surfactant, a reductant, and a solvent wherein a reduction reaction is performed in said mixture solution to form a metal nanoparticle; and
adding a second metal precursor after said metal nanoparticle is formed, to have the number of particles of said second metal precursor be more than the total number of said metal nanoparticles;
wherein the concentration difference between said metal nanoparticle and said second metal precursor results in a non-equilibrium coexistence system and then said metal nanoparticle breaks down to metal nanoclusters with a smaller particle diameter so as to form an equilibrium system.

20. The method according to claim 19, wherein said first metal precursor is selected from the group consisting of the following: AuCl3, HAuCl4, AuBr3, and HAuBr4.

21. The method according to claim 19, wherein said second metal precursor is selected from the group consisting of the following: AuCl3, HAuCl4, AuBr3, and HAuBr4.

22. The method according to claim 19, wherein said first metal precursor and said second metal precursor are the same.

23. The method according to claim 19, wherein said first metal precursor and said second metal precursor are different.

24. The method according to claim 19, wherein said surfactant is selected from the group consisting of the following or combination thereof: didodecyldimethylammonium bromide (DDAB), tetraoctylammonium bromide (TOAB), and tetrabutylammonium bromide (TBAB).

25. The method according to claim 19, wherein said reductant is selected from the group consisting of the following or combination thereof: tetrabutylammonium borohydride (TBAB), NaBH4, and ascorbic acid.

26. The method according to claim 19, wherein said solvent is selected from the group consisting of the following or combination thereof: toluene and chloroform.

27. The method according to claim 19, wherein said metal nanoparticle comprises a property of surface plasmon absorption.

28. The method according to claim 19, wherein the particle diameter of said metal nanocluster is between 1 nm and 4 nm.

29. The method according to claim 19, wherein a ligand-binding reaction is performed, after said metal nanocluster is formed, to bind a ligand to the surface of said metal nanocluster so as to form a ligand-capped metal nanocluster.

30. The method according to claim 29, wherein said ligand is selected from the group consisting of the following: dihydrolipoic acid (DHLA), dodecanethiol (DDT), Bis(p-sulfonatophenyl)phenylphosphine (BSPP), and triphenylphosphine.

31. The method according to claim 29, wherein said ligand-binding reaction is a thiol-related ligand binding reaction to bind a thiol-related ligand to the surface of said metal nanocluster so as to form a thiol-capped metal nanocluster.

32. The method according to claim 29, wherein said thiol-related ligand is selected from the group consisting of the following: dihydrolipoic acid (DHLA), dodecanethiol (DDT), meso-2,3-dimercaptosuccinic acid (DMSA), glutathione (GSH), and 1,6-hexanedithiol.

33. The method according to claim 31, wherein said thiol-capped metal nanocluster is a fluorescent metal nanocluster.

34. The method according to claim 33, wherein the particle diameter of said fluorescent metal nanocluster is between 0.5 nm and 3 nm.

35. The method according to claim 33, wherein a functional coating reaction is performed, after said fluorescent metal nanocluster is formed, to have said fluorescent metal nanocluster comprise a functional group.

36. The method according to claim 35, wherein the functional group of said functional coating reaction is selected from the group consisting of the following: chemical functional group, crosslinking molecule, saccharide, fluorescent molecule, paramagnetic molecule, bio-molecule, and drugs.

37. The method according to claim 35, wherein said functional coating reaction is a bioconjugation reaction.

38. The method according to claim 35, wherein said fluorescent metal nanocluster is used as bioprobes and/or applied in fluorescent biological label, clinical image as contrast medium, clinical detection, clinical trace, and clinical treatment.
